(19) United States
(12) Patent Application Publication
(10) Pub. No.: US 2022/0211329 A1
(43) Pub. Date: Jul. 7, 2022

(54) METHOD AND SYSTEM FOR ENHANCING GLUCOSE PREDICTION

(71) Applicant: UNIVERSITAT POLITÈCNICA DE VALÈNCIA (UPV), Valencia (ES)

(72) Inventors: Jose Luis DIEZ RUANO, Valencia (ES); Jorge BONDÍA COMPANY, Valencia (ES); Eslam MONTASER ROUSHDI ALI, Valencia (ES)

(21) Appl. No.: 17/143,572

(22) Filed: Jan. 7, 2021

(51) Int. Cl. A61B 5/00; A61B 5/145; G16H 40/63; G06N 5/02

(52) U.S. Cl. CPC A61B 5/7275; A61B 5/14532; A61B 5/4866; A61B 5/7225; A61B 5/7267; A61B 5/7203; G16H 40/63; G06N 5/02; A61B 5/7264

(57) ABSTRACT

A method for predicting blood glucose based on seasonal local models, which enhances glucose prediction allowing to control the glucose level more precisely, and comprises the steps of providing raw data time series, comprising a record of measured blood glucose data; preprocessing the raw data time series for obtaining event periods by dividing the raw data time series into event periods, by setting timestamps of main meal events and enforcing seasonality of event periods by expanding fictitiously; clustering event periods by using techniques for clustering incomplete data, which partitions data into c cluster prototypes, identifying a set of c seasonal local models, predicting the blood glucose for a desired prediction horizon by using the seasonal local models, integrating the local glucose predictions for obtaining a real-time BG prediction through real-time membership-to-cluster estimation; and saving BG prediction Ĝ(t|tₚ), t in [tₚ,tₚ+PH].

### OBJECT OF THE INVENTION

The present invention is framed in the technological field of glucose prediction and control. Specifically, the invention is directed to a more precise monitorization of the glucose concentration values in humans.

An object of the present invention is to provide a method for enhancing glucose prediction which allows to control the glucose level more precisely by taking into account new control parameters.

A second object of the present invention is to provide a system for enhancing glucose prediction configured to carry out the method of the invention.

### BACKGROUND OF THE INVENTION

Diabetes represents one of the major health challenges of the 21st century. It is estimated that 463 million people worldwide had diabetes in 2019, and by 2045 this number may rise to 700 million. About 10% of people with diabetes suffer from type 1 diabetes (T1D), the most severe kind. T1D is characterized by autoimmune destruction of the β-cells in the pancreas, responsible for the secretion of the hormone insulin, an essential hormone needed to maintain blood glucose (BG) concentrations in a narrow range (70-140 mg/dL).

People with T1D need to replace endogenous insulin secretion with life-long subcutaneous exogenous insulin administration by multiple daily injections or continuous infusion via a portable pump, to avoid the detrimental effects of hyperglycemia. Good BG control can help diabetic patients to prevent or delay serious long-term complications of diabetes. Therefore, T1D patients must monitor their BG levels throughout the day and night to avoid BG problems such as hyperglycemia or hypoglycemia.

Nowadays, it is possible to continuously monitor the glucose level and its trends by using continuous glucose monitoring (CGM) systems. A CGM basically consists of a sensor measuring interstitial glucose levels, and transmitting information to an external display device able to provide alerts to the patient if the glucose level is reaching certain thresholds. In sensor pump integrated systems, CGM can also send the quasi-time-continuous readings to an insulin pump that can take actions depending on glucose levels.

The technological advances in diabetes treatment with the advent of CGMs have paved the way for a fully automatic treatment regime, automatic insulin delivery. It is then referred to as the “Artificial Pancreas” (AP) system, a closed-loop system where a control algorithm decides the proper insulin infusion in a continuous-time manner.

Therefore, the main idea through the AP system is to connect the CGM with the insulin pump via a control algorithm to compute the optimal insulin dose administered through an insulin pump to prevent T1D patients from having hypoglycemia or hyperglycemia and keep their BG concentration in an acceptable range (70-180 mg/dL), based on information received from a CGM every 5 minutes.

CGMs have also paved the way to decision support systems (DSS) when connected with an insulin pump or smart insulin pen, providing automated data analytics useful to the patient for a better diabetes self-control.

The ability to predict glucose along a given prediction horizon (PH), and estimation of future glucose trends, is the most important feature of any AP and DSS, in order to be able to take preventive actions to entirely avoid risk to the patient. Glucose prediction can appear in an AP as part of the control algorithm itself, such as in systems based on model predictive control (MPC) techniques, or as part of a monitoring system to avoid hypoglycemic episodes. In a DSS, glucose prediction allows to inform the patient about risks, predict hypoglycemic and hyperglycemic events, detect abnormal patient glucose responses, and take better informed decisions for improved glucose control. Therefore, high-reliability glucose prediction models have the potential to significantly improve diabetes management.

“Seasonal” stochastic time series models present regular patterns of changes and fluctuations that repeat periodically such as SARIMA: Seasonal AutoRegressive Integrated Moving Average, or SARIMAX: SARIMA including eXogenous variables. When trained from fixed-length clustered postprandial (after meal) glucose time series, they have exhibited relatively higher BG prediction accuracy for long term PHs and achieved both lowest root-mean-square error (RMSE) and mean absolute percentage error (MAPE) for different PHs when compared to other solutions, including nonlinear solutions, such as, recurrent neural networks and nonlinear regression.

Despite SARIMA and SARIMAX models have demonstrated previously their effectiveness for glucose prediction in long PHs with high accuracy, fixed-length time series requirements do not allow these methods to be applied in normal life (i.e. free-living conditions with the support of CGM data), which includes a variety of event-related periods (meals, nights, exercise, hypoglycemia treatments, etc.) with different and variable lengths.

### DESCRIPTION OF THE INVENTION

The invention is related to a method for prediction of blood glucose based on seasonal local models. The method of the invention improves the accuracy of the blood glucose prediction by integrating a set of local models with a forced seasonality trained from clustered incomplete time series data.

Accurate predictions of blood glucose (BG) concentration with longer prediction horizon (PH) might improve type 1 diabetes therapy by allowing artificial pancreas (AP) and decision support systems (DSS) to adjust the therapy based on BG future values.

The method of the invention allows to predict blood glucose by integrating the predictions of a number of seasonal local models, “local” referring to each seasonal model representing a data set of similar glucose profiles observed along CGM historical data.

In the modeling step, the number of data sets and their corresponding glucose profiles characteristics (prototypes) are obtained by techniques for clustering of incomplete data (for instance, Partial Distance Strategy Fuzzy C-Means). Then, it is identified a seasonal model for each data set, for example using a Box-Jenkins methodology. Finally, the online BG prediction is obtained by local model integration through real-time membership-to-cluster estimation.

Firstly, raw historical data time series are provided, which comprises a record of measured blood glucose (BG) data from a continuous glucose monitor (CGM), and timestamps of main meal events. Optionally, additional data can be considered:
Timestamps of additional events including time of hypoglycemia treatment and time of start of an exercise session.
Labels identifying the nature of such events (for example, “meal”, “breakfast”, “lunch”, “dinner”, “night”, “hypoglycemia treatment”, “exercise”, “missed bolus”, etc.).
Time series of exogenous inputs including insulin infusion and physiological signals from, for example, a physical activity monitor (heart rate, energy expenditure, skin temperature, galvanic skin response, etc.)
   Said data is treated as an input for constructing the local models.

The method of the invention preprocesses original CGM historical data under free-living conditions (normal life) to obtain sets of similar glycemic profiles (clusters) useful for seasonal model identification of different event periods. This CGM data can be from a single patient, producing an individualized model where clusters characterize intra-patient variability, or from a population of patients, providing a population model where clusters characterize intra- and inter-patient variability.

For that, the raw CGM data time series and events timestamps are preprocessed for obtaining a set of event periods. For that, the raw data CGM time series are split into said event periods. An event period starts at the timestamp of said event and ends on the timestamp of the next event. In the particular event period after last meal in a day, the period event is split in two period events. Preferably, the first one having a fixed duration, then, starting the second one after said duration is finished and ending on the first event of the next day. This second period is used to represent a nocturnal period. The set of event periods can be divided into different subsets using event labels when available (for instance, subset 1 collecting breakfast, lunch and dinner event periods, subset 2 collecting night event periods, subset 3 collecting hypoglycemia treatments event periods, etc.). When available, exogenous inputs time series are also split in said subsets. In this case, each subset will be clustered separately.

Having being obtained the subset of event periods, length of said event periods is regularized. This is achieved by expanding fictitiously the event periods until the duration of each event period is equal to the duration of the period event having the maximum duration (s). For that, “not a number” (NaN) values are added after the last measured BG value and exogenous inputs (when available), producing time series with incomplete data.

Then, the event periods are clustered by a clustering algorithm for incomplete data, preferably using a Partial Distance Strategy Fuzzy C-Means clustering (PDSFCM) algorithm. Therefore, the events periods belongs partially to c clusters, represented by their prototypes (centroids), according to a fuzzy membership depending on the distance of said event periods to said cluster prototypes. Preferably, an optimal number of clusters (c) is obtained by using an index taking into account clusters cohesion and separation, such are Xie-Beni index (XBI) or Fukuyama-Sugeno index (FSI). In one embodiment, the clustering step is carried out by PDSFCM algorithm, which computes the cluster prototypes and membership values by minimizing the equation:

$${J}_{m} ( U , V ; X ) = {\sum }_{i = 1}^{c} {\sum }_{j = 1}^{n} {u}_{ij}^{m} {d}_{ij}^{2} ( {x}_{j} , {v}_{i} ) \qquad Eq . 1$$

In Eq. 1, X={x₁, x₂, . . . , xₙ} denotes regularized-length event periods extracted from raw data time series, V=(v₁, v₂, . . . , v_{c}) is a vector of unknown cluster prototypes vᵢ∈ [symbol] ^{p}, d is a distance function representing the partial distance between the event periods (incomplete time series) and cluster prototypes and m ∈[1, ∞) is a fuzziness parameter, U=[uᵢⱼ] with i=1, 2, . . . , c, and, j=1, 2, . . . , n is a matrix of memberships wherein:
uᵢⱼ∈[0, 1];
Σᵢ₌₁^{c}uᵢⱼ=1, ∀j; and
0<Σⱼ₌₁ⁿuᵢⱼ<n, ∀i.

The clustering step does not affect modeling as far as it is a data preprocessing step, and this process cannot cause overfitting, i.e. the process does not introduce additional unnecessary parameters, which avoid the generalization of the model.

Then, a prediction model of the blood glucose is calculated, by modeling data in each cluster by a “local model” to simplify the glucose dynamic system, building for example a SARIMA (or SARIMAX if exogenous input time series are included) local model for each cluster, thus, obtaining a set of local models providing a set of glucose predictions.

The concept of seasonality has demonstrated successfully its accuracy in the BG prediction models for long PHs, but it is not directly applicable for the use in the normal life. Enforcing the concept of seasonality in normal life data then is very useful for developing BG prediction models or for detecting abnormal behaviors of people with diabetes.

First, for each cluster a time series is obtained by concatenating, time ordered, the event periods in said cluster, or a subset of them with a membership value (μ) to said cluster above a given threshold μₘᵢₙ=0. Glucose values previous to the event timestamp (hence referred as presampling data) are also saved in a number given by the maximum expected order of the AR component of the to-be-identified local models. Presampling data will serve to properly initialize the local models.

Each SARIMA model for each time series is defined by the equations:

G(t)=α+w(t) Eq. 2

ϕₚ(z⁻¹) [symbol] _{P}(z^{-s})∇ₛ^{D}∇^{d}w(t)=θ_{q}(z⁻¹)θ_{Q}(z^{-s})ε(t) Eq. 3

In Eq. 2, G(t) is the glucose concentration at time t, α is a constant term, called intercept, ∇ is the backward difference operator, defined as:

∇w(t)=w(t)−w(t−1)

Also, d is the non-seasonal integration order of the time series, D is the seasonal integration order of the time series, ∇ₛ is the seasonal backward difference operator, defined as:

∇ₛw(t)=w(t)−w(t−s)

Also, ε(t) is an stochastic error following a white noise process defined by:

ε(t)˜WN(0, σ²)

And ϕₚ(z⁻¹), [symbol] _{P}(z^{-s}), θ_{q}(z⁻¹) and θ_{Q}(z^{-s}) are polynomials in the lag (back-shift) operator z⁻¹ of degree p, q, P, and Q, respectively, defined as:

ϕₚ(z⁻¹)=1−ϕ₁z⁻¹−ϕ₂z⁻²− . . . −ϕₚz^{-p} Eq. 4

[symbol]

_{P}(z^{-s})=1− [symbol] ₛz^{-s}− [symbol] ₂ₛz^{-2s}− . . . − [symbol] _{Ps}z^{-Ps} Eq. 5

θₚ(z⁻¹)=1+θ₁z⁻¹+θ₂z⁻²+ . . . +θ_{q}z^{-q} Eq. 6

θ_{Q}(z^{-s})=1+θₛz^{-s}+θ₂ₛz^{-2s}+ . . . +θ_{Qs}z^{-Qs} Eq. 7

When exogenous inputs are considered, each SARIMAX model for each cluster time series is defined by the equations:

$$\begin{matrix}G ( t ) = α + {\sum }_{i = 1}^{{n}_{x}} {η}_{i , {r}_{i}} ( {z}^{- 1} ) {X}_{i} ( t ) + w ( t ) & Eq . 8 \\ {ϕ}_{p} ( {z}^{- 1} ) {ϕ}_{p} ( {z}^{- s} ) {∇}_{S}^{D} {∇}^{d} w ( t ) = {θ}_{q} ( {z}^{- 1} ) {θ}_{Q} ( {z}^{- s} ) ɛ ( t ) & Eq . 9\end{matrix}$$

where nₓ is the number of exogenous inputs, Xᵢ is the exogenous input i at time t and η_{i,r}(z⁻¹), i=1, . . . , nₓ are polynomials in the lag operator z⁻¹ of degree rᵢ, defined as:

η_{i,r}_{ᵢ}(z⁻¹)=η_{i,0}+η_{i,1}z⁻¹+η_{i,2}z⁻²+ . . . +η_{i,r}_{ᵢ}z^{-r}^{ᵢ} Eq. 10

Alternatively, in Eqs 2-3 and 8-9, the difference operators ∇ and ∇ₛ can be applied to the signal G(t), instead of w(t). Thus, a SARIMA model can also be represented as

∇ₛ^{D}∇^{d}G(t)=α+w(t) Eq. 2′

ϕₚ(z⁻¹) [symbol] _{P}(z^{-s})w(t)=θ_{q}(z⁻¹)θ_{Q}(z^{-s})ε(t) Eq. 3′

And a SARIMAX model as

$$\begin{matrix}{∇}_{S}^{D} {∇}^{d} G ( t ) = α + {\sum }_{i = 1}^{{n}_{x}} {η}_{i , {r}_{i}} ( {z}^{- 1} ) {X}_{i} ( t ) + w ( t ) & Eq . {8}^{′} \\ {ϕ}_{p} ( {z}^{- 1} ) {ϕ}_{p} ( {z}^{- s} ) w ( t ) = {θ}_{q} ( {z}^{- 1} ) {θ}_{Q} ( {z}^{- s} ) ɛ ( t ) & Eq . {9}^{′}\end{matrix}$$

Preferably, the identification of an optimal seasonal model for each time series is carried out by means of the Box-Jenkins methodology which indicates the steps for identifying, estimating, and checking time series models: checking for stationarity or non-stationarity and differencing the data, if necessary; identifying and selecting an appropriate model structure; estimating the parameters of the chosen model; diagnostic checking of the chosen model; and forecasting, and re-identifying a new model if necessary.

Being obtained the set of SARIMA (or SARIMAX) glucose models, an integration step is performed for obtaining a real-time BG prediction for a desired PH, by a time-varying weighting of the set of glucose predictions given by the seasonal local models, defined by the equations:

$$\hat{G} ( t \vert {t}_{p} ) = {\sum }_{i = 1}^{c} {γ}_{i} ( {t}_{p} ) {\hat{G}}_{i} ( t \vert {t}_{p} ) \qquad Eq . 11$$

where tₚ is the time at which the prediction is computed, t is the time of the predicted value, in the time interval [tₚ,tₚ+PH], Ĝᵢ(t|tₚ) is the predicted glucose by local model i, for i={1, 2, . . . , c}, γᵢ(tₚ) is a fuzzy membership corresponding to the weight of model i at time tp, and Ĝ(t|tₚ) is the final predicted glucose value for future time t, computed at current time tₚ.

Time-varying weights γᵢ(tₚ), i=1, . . . , c, are computed from the membership value of CGM measurements to each cluster i, with respect to a time window [t₀,t₁], denoted as uᵢ(t₁; t₀), and defined by:

$${u}_{i} ( {t}_{1} ; {t}_{0} ) = \frac{1}{{\sum }_{i = 1}^{c} {( \frac{{d}^{2} ( {[ G ]}_{{t}_{0}}^{{t}_{1}} ( t ) , {[ {v}_{i} ]}_{{t}_{0}}^{{t}_{1}} ( t ) )}{{d}^{2} ( {[ G ]}_{{t}_{0}}^{{t}_{1}} ( t ) , {[ {v}_{i} ]}_{{t}_{0}}^{{t}_{1}} ( t ) )} )}^{\frac{1}{m - 1}}} \qquad Eq . 12$$

where [G]ₜ_{₀}^{t}^{₁}(t) is the segment of CGM data from t₀ to t₁, [vᵢ]ₜ_{₀}^{t}^{₁}(t) is the segment of the prototype for cluster i from t₀ to t₁, and d is the distance measured.

In one embodiment, the time window [t₀,t₁] is defined as the time interval from the timestamp of the last event (tₑₖ) up to current time (tₚ), so that

γᵢ(tₚ)=uᵢ(tₚ;tₚ−tₑₖ) Eq. 13

In one embodiment, time-varying weights γᵢ(tₚ), i=1, . . . , c, are computed in a two-step procedure, In the first step, clusters with a high enough contribution in a first time window [tₚ−t_{w1},tₚ] are selected, as defined by the index set

I={i|uᵢ(tₚ;tₚ−t_{w1})≥μₘᵢₙ(tₚ)} Eq. 14

Preferably t_{w1}=tₑₖ is chosen. In a second step, selected clusters contributions are computed, with respect a second shorter time window [tₚ=t_{w2},tₚ], so that t_{w2}<t_{w1}, as defined by:

$${γ}_{i} ( {t}_{p} ) = \{ \begin{matrix}{u}_{i} ( {t}_{p} ; {t}_{p} - {t}_{W 2} ) & for i ∈ I \\ 0 & otherwise\end{matrix} \qquad Eq . 15$$

Finally, the BG prediction Ĝ(t|tₚ), t in [tₚ,tₚ+PH], obtained is saved.

The integration step for obtaining a real-time BG prediction is preferably carried out by performing the following phases:
a) receiving a new event time stamp, and optionally, an event type label;
b) receiving a CGM measurement, and optionally, exogenous inputs measurements;
c) If the event is labelled, selecting the set of c clusters and corresponding local models for that event type, obtained in the clustering and modeling steps. If the events are not labelled, there will be a unique set of c clusters and local models;
d) calculating the membership to the selected set of c clusters of the CGM measurements up to current time for the current event period;
e) combining the predictions of the c local models using the memberships calculated in step d);
f) repeating steps d) and e) every new CGM measurement until the period is finished (the next event timestamp is received);
g) adding the finished event period as a new element of the cluster having the highest membership, and the historical data of the corresponding local model; and
h) repeating the previous steps with the new event period

The method of the invention could also comprise a step of calculating a crispness index, which gives a measure of how much the glucose prediction is produced by a single model. The glucose model is a multi-model system, but is more reliable if one of the available local models match perfectly. Therefore, fuzziness is included to give robustness by the integration of the local models when one of them is not enough to provide the best estimation.

For obtaining the crispness index at time tₚ, the Manhattan distance, normalized to the interval [0,1], between the vector composed by the membership values to the c clusters and the equally-distributed-membership vector (1/c, 1/c, . . . , 1/c) representing the lowest crispness (maximum fuzziness) is used, defined as:

$$CI ( {t}_{p} ) = \frac{1}{2 ( 1 - \frac{1}{c} )} {\sum }_{i = 1}^{c} \vert {γ}_{i} ( {t}_{p} ) - \frac{1}{c} \vert \qquad Eq . 16$$

The method of the invention could also comprise a step of calculating a normality index, which gives a measure of how much the measured CGM data in the current event period is similar to historical data represented by the clusters.

The normality index at time tₚ is calculated by:
calculating possibility memberships so that
   uᵢⱼ^{P}∈[0, 1]; and
   0<Σⱼ₌₁ⁿuᵢⱼ^{P}<n, ∀i.
summing up the possibility memberships, and normalizing to the interval [0,1]
determining the normality degree of the period:
   if the sum of normalized possibility memberships is greater than a defined threshold (thr_{Nl}), then it is normal,
   if the sum of normalized possibility memberships is lower than thr_{Nl}, then it is abnormal.

Therefore, the proposed system allows diabetic patients to detect abnormal states which can guide therapeutic decisions.

The invention refers also to a system for controlling blood glucose automatically, which comprises one CGM sensor; and a control unit, connected to the CGM sensor.

The control unit of the system is adapted to perform the steps of the method of the invention explained.

The system of the invention could further comprise a pump for delivering insulin, which is connected to the control unit. The control unit in this case would be configured to calculate the amount of insulin to provide according to the calculated BG prediction.

Also the system could comprise a communication module connected to the control unit, to connect to an external device. In this case, the control unit would be configured to calculate the cripness index following the steps explained for the method of the invention. Having obtained the cripness index, the communication module sends said cripness index to the external device.

In the same way, the system could also comprise an alert module connected to the control unit, to activate an alarm when an abnormal glucose behavior is detected. In this case, the control unit is configured to calculate a normality index following the steps explained for the method of the invention. Then, the alert module activates the alarm when the calculated normality index is abnormal.

### DESCRIPTION OF THE DRAWINGS

To complement the description being made and in order to aid towards a better understanding of the characteristics of the invention, in accordance with a preferred example of practical embodiment thereof, a set of drawings is attached as an integral part of said description wherein, with illustrative and non-limiting character, the following has been represented:

FIG. 1.—shows a flow chart of a preferred embodiment of the method of the invention from raw data to the concatenating step.

FIG. 2.—Shows a diagram representing the processing of the raw data according to the preferred embodiment of the invention.

FIG. 3.—shows a flow chart of a preferred embodiment of the method of the invention for the modeling step.

FIG. 4.—shows a flow chart of the validation step of the preferred embodiment of the invention.

FIG. 5.—shows a diagram of a validation time series of the preferred embodiment of the invention.

FIG. 6.—shows a flow chart of a preferred embodiment of the method of the invention for the local model integration step for real-time glucose prediction. This is referred as “global seasonal model” (GSM).

FIG. 7.—shows an example of GSM to demonstrate the relation between predictions, fuzzy memberships, crispness, and normality indexes. In this case, 120-min-ahead predictions are performed for a meal event at t=0, t=60, t=120 and t=180. In the upper panel, local model predictions (LM1 to LM5) are showed in thin solid line. Global prediction and actual glucose are shown as explained by the legend. Local model weights γᵢ(tₚ) computed following the two-step integration method (fuzzy memberships), crispness index and normality index, are shown in the next panels, computed at each time instant up to 120 minutes before the end of the meal event.

FIG. 8.—shows another example of how the system behaves in the case of abnormal behaviors (missed bolus at a meal). In this case, 120-min-ahead predictions are performed for a meal event at t=0, t=60, t=120 and t=180. In the upper panel, local model predictions (LM1 to LM5) are shown. Global prediction and actual glucose are shown as explained by the legend. Local model weights γᵢ(tₚ) computed following the two-step integration method (fuzzy memberships), crispness index and normality index, are shown in the next panels, computed at each time instant up to 120 minutes before the end of the meal event. Low normality indicates this response is very dissimilar to historical data.

FIG. 9.—shows an example of abnormal states detection. Marked boxes indicate the periods detected as abnormal, corresponding to a normality index below or equal to 0.2. Shaded box indicates a postprandial period with a missed bolus. Meshed box indicate an exercise session including the 3-hour period post-exercise with altered insulin sensitivity. Missed boluses and exercise were not considered in the models training data. Sustained hyperglycemia due to the missed bolus, as compared to a regular meal, is detected as an abnormal period. Rapid decline of glucose due to the exercise and posterior dinner response (with altered insulin sensitivity and mismatch insulin bolus) is also detected as abnormal state. Additionally a period around sample 6300 is also detected as abnormal, corresponding to an unusual sustained glucose close to hypoglycemia in a late postprandial response, resulting from response variability.

### PREFERRED EMBODIMENT OF THE INVENTION

The method of the invention preprocesses original continuous glucose monitoring (CGM) historical data under free-living conditions (normal life) to obtain sets of similar glycemic profiles (clusters) useful for seasonal model identification of different event periods.

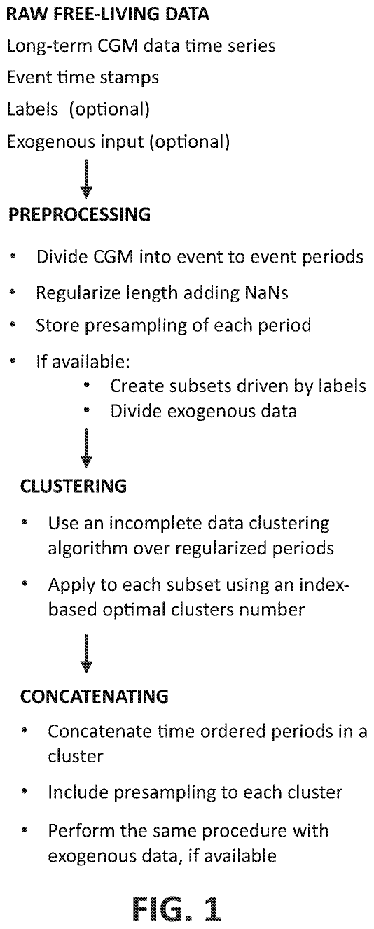

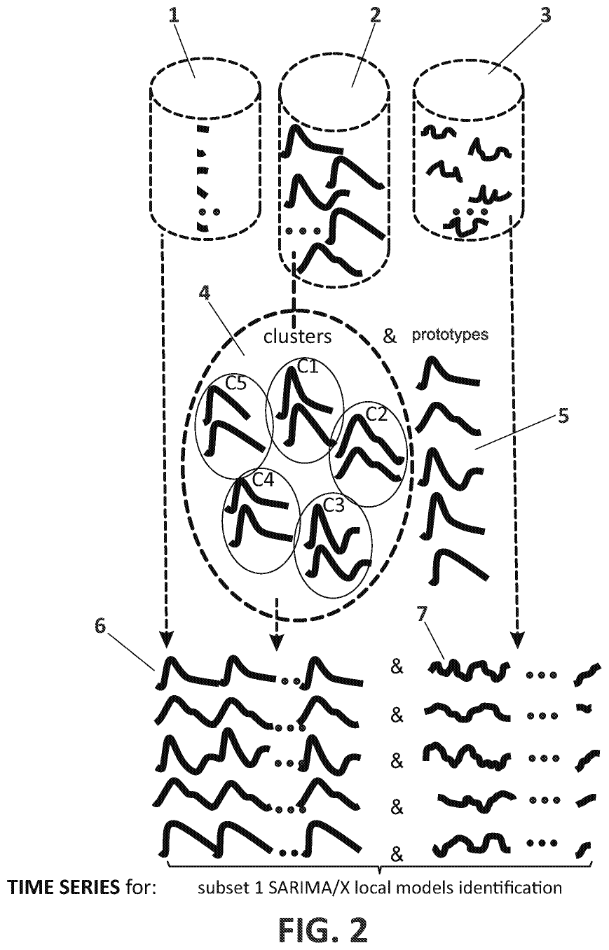

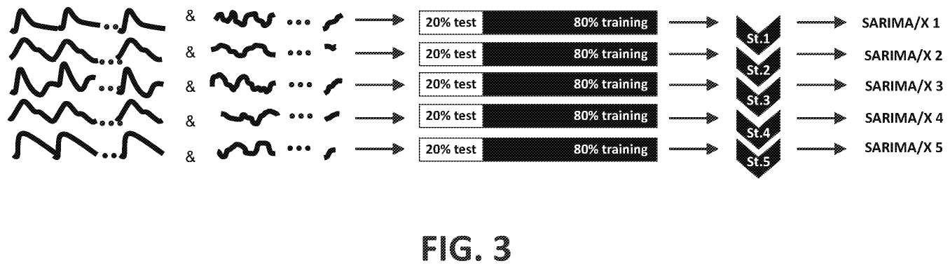

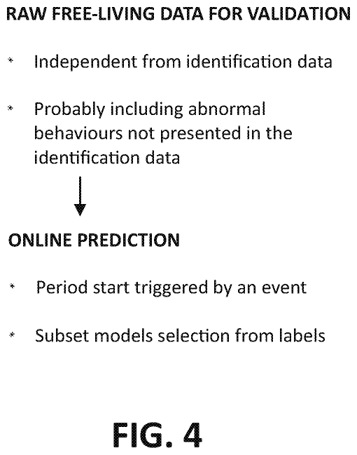

FIGS. 1 to 6 show a flow chart of a preferred embodiment of the method of the invention. FIGS. 1 and 2 show the CGM data preprocessing and clustering step. FIG. 3 shows the local models identification step. FIGS. 4, 5 and 6 show the real-time computation of glucose predictions and validation procedure.

First of all, original CGM historical data of different full days, including timestamps of meals, hypoglycemia treatment, and other events of interest, will be preprocessed. The long-term data is partitioned into a set of “event-to-event” time subseries, called event periods, driven by said events timestamps, to enforce seasonality, being original periods in different lengths.

Seasonality is enforced to apply the seasonal stochastic modeling techniques. The initial instant, duration, and final instant of each event could be variable, since original time series data is partitioned into a set of “event-to-event” time subseries, driven by event timestamps. Therefore, each time subseries initial point is the time instant when the event starts and its final point is the initial point of the next event. A special case is the after dinner where a fixed-length (for example, 6 hours) postprandial period is considered, then, the night period starts, finalizing at breakfast time. The subset of event periods can be divided into different subsets using event labels when available (for instance, subset 1 collecting breakfast, lunch and dinner event periods, subset 2 collecting night event periods, subset 3 collecting hypoglycemia treatments event periods, etc.). When available, exogenous inputs time series are also split in said subsets. In this case, each subset will be clustered separately.

Hence, each day provides at least four subseries with different lengths (three main meals and night period), and these lengths change every day. In order to force seasonality, the period with the maximum duration is detected and its length is stored as “s”. Then, all the subseries duration is fictitiously expanded to “s”, by adding “not a number -NaN-” values after the last available value, giving rise to regularized-length incomplete (missing data) time series data. In this way, all the periods will have the same length (s) but most of them will have some NaNs in the final positions of the time subseries.

Preprocessed data are then clustered and, once the c (number of clusters) sets of similar enough event periods with the same (enforced) length, resulting from the said partitioning of the original CGM time series, are available as training sets, each cluster is modeled as a SARIMA (or SARIMAX) “local model” with seasonality period s to simplify the glucose dynamic prediction, leading to a set of local models for glucose prediction Ĝᵢ(t) for i={1, 2, . . . , c}. Finally, the BG prediction is obtained by integrating the local models predictions.

For clustering the preprocessed data, a clustering algorithm, for example fuzzy C-Means (FCM) algorithm, is used. Nevertheless, FCM algorithm cannot be directly applied to incomplete data (missing data), since all data is required to compute the cluster prototypes (centroids) and the distance measures.

Therefore, a Partial Distance Strategy FCM (PDSFCM) algorithm is used to clustering event periods incomplete data. PDSFCM partitions data into c>1 clusters, wherein the optimal number of clusters could be determined automatically by using an index taking into account clusters cohesion and separation, such are Xie-Beni index (XBI) or Fukuyama-Sugeno index (FSI) index, by minimizing the following objective function:

$${I}_{m} ( U , V ; X ) = {\sum }_{i = 1}^{c} {\sum }_{j = 1}^{n} {u}_{ij}^{m} {d}_{ij}^{2} ( {x}_{j} , {v}_{i} ) \qquad Eq . 1$$

In Eq. 1, X={x1, x2, . . . , xn} denotes regularized-length event periods extracted from raw data time series, V=(v1, v2, . . . , vc) is a vector of unknown cluster prototypes vᵢ∈ [symbol] ^{p}, d is a distance function representing the partial distance between the event periods (incomplete time series) and cluster prototypes and m ∈[1,∞) is a fuzziness parameter, U=[uᵢⱼ] with i=1, 2, . . . , c, and, j=1, 2, . . . , n is a matrix of memberships wherein:
uᵢⱼ∈[0, 1];
Σᵢ₌₁^{c}uᵢⱼ=1, ∀j; and
0<Σⱼ₌₁ⁿuᵢⱼ<n, ∀i.

FIG. 2 shows a representation of said exemplary embodiment. Regularized periods (2) are clustered in 5 clusters (4). Some prototypes (5) are also generated. Then, presampling periods (1), clusters (4) and prototypes (5) are concatenated (6). Then, exogenous data (3) are added (7) to the concatenated data (6).

Then, data in each cluster is modeled as a SARIMA (or SARIMAX) “local model”, which is an expanded form of its non-seasonal counterpart ARIMA model that includes as new model components seasonal autoregressive (SAR) and seasonal moving average (SMA) terms. SAR and SMA terms are added so that an observation at time t depends on: previous observations and previous stochastic errors. In both cases, the previous values are taken at times with lags that are multiples of the seasonality period s, as detailed in equations (2)-(7). This means that predicted values will not only depend on recent past values, but also on behaviors observed in recent events in the same cluster.

First, for each cluster a time series is obtained by concatenating, time ordered, the event periods in said cluster, or a subset of them with a membership value to said cluster above a given threshold μₘᵢₙ>0.

Given a concatenated CGM time series {G(t)|t=1, 2, . . . , k} resulting from the clustering of regularized-length event periods, a SARIMA model is expressed as:

G(t)=α+w(t) Eq. 2

ϕₚ(z⁻¹) [symbol] _{P}(z^{-s})∇ₛ^{D}∇^{d}w(t)=θ_{q}(z⁻¹)θ_{Q}(z^{-s})ε(t) Eq. 3

In Eq. 2, G(t) is the glucose concentration at time t, α is a constant term, called intercept, ∇ is the backward difference operator, defined as:

∇w(t)=w(t)−w(t−1)

Also, d is the non-seasonal integration order of the time series, D is the seasonal integration order of the time series, ∇s is the seasonal backward difference operator, defined as:

∇ₛw(t)=w(t)−w(t−s)

Also, ε(t) is an stochastic error following a white noise process defined by:

ε(t)˜WN(0, σ²)

And ϕₚ(z⁻¹), [symbol] _{P}(z^{-s}), θ_{q}(z⁻¹) and θ_{Q}(z^{-s}) are polynomials in the lag (back-shift) operator z−1 of degree p, q, P, and Q, respectively, defined as:

ϕₚ(z⁻¹)=1−ϕ₁z⁻¹−ϕ₂z⁻²− . . . −ϕₚz^{-p} Eq. 4

[symbol]

_{P}(z^{-s})=1− [symbol] ₛz^{-s}− [symbol] ₂ₛz^{-2s}− . . . − [symbol] _{Ps}z^{-Ps} Eq. 5

θₚ(z⁻¹)=1+θ₁z⁻¹+θ₂z⁻²+ . . . +θ_{q}z^{-q} Eq. 6

θ_{Q}(z^{-s})=1+θₛz^{-s}+θ₂ₛz^{-2s}+ . . . +θ_{Qs}z^{-Qs} Eq. 7

Each model can be expressed in a short form as SARIMA (p, d, q)(P,D, Q)ₛ. The SAR, and SMA coefficients can be estimated by fitting SAR and SMA models to the residual errors ε(t) themselves.

Clustering event periods, previously enforcing seasonality to a period “s” equal to the length of the original longest period, would provide similar enough event periods as training and validation sets, for determining a SARIMA model for each cluster i, preferably by using a Box-Jenkins methodology, leading to a set of local glucose predictions Ĝᵢ(t|tₚ) for i={1, 2, . . . , c}, for a desired prediction horizon (PH) at time instant (tₚ).

Then, a local models integration is performed by a fuzzy approach, for obtaining a real-time BG prediction Ĝ(t|tₚ)) for a desired prediction horizon (PH) at time instant (tₚ), wherein the available (CGM previous to tₚ) data belongs partially (from 0 to 1) to all clusters and the BG prediction is computed by weighting the c estimations according to the fuzzy membership (γᵢ(tₚ)) at time tₚ to each cluster i. The integration step is defined by the equations:

$$\begin{matrix}\hat{G} ( t \vert {t}_{p} ) = {\sum }_{i = 1}^{c} {γ}_{i} ( {t}_{p} ) {\hat{G}}_{i} ( t \vert {t}_{p} ) & Eq . 11 \\ {u}_{i} ( {t}_{1} ; {t}_{0} ) = \frac{1}{{\sum }_{i = 1}^{c} {( \frac{{d}^{2} ( {[ G ]}_{{t}_{0}}^{{t}_{1}} ( t ) , {[ {v}_{i} ]}_{{t}_{0}}^{{t}_{1}} ( t ) )}{{d}^{2} ( {[ G ]}_{{t}_{0}}^{{t}_{1}} ( t ) , {[ {v}_{i} ]}_{{t}_{0}}^{{t}_{1}} ( t ) )} )}^{\frac{1}{m - 1}}} & Eq . 12 \\ I = \{ i \vert {u}_{i} ( {t}_{p} ; {t}_{p} - {t}_{W 1} ) \geq {μ}_{min} ( {t}_{p} ) \} & Eq . 14 \\ {γ}_{i} ( {t}_{p} ) = \{ \begin{matrix}{u}_{i} ( {t}_{p} ; {t}_{p} - {t}_{W 2} ) & for i ∈ I \\ 0 & otherwise\end{matrix} & Eq . 15\end{matrix}$$

In Eq. 8,γᵢ(tₚ) is the fuzzy membership at time tₚ, i.e. the weight of each SARIMA model in predicting the Ĝᵢ(t|tₚ) for i={1, 2, . . . , c} is a set of glucose predictions given by the local models for each cluster. Fuzzy membership γᵢ(tₚ) is computed in two steps. In the first step, fuzzy membership of the CGM in the time window [tₚ−t_{w1},tₚ] is computed in Eq. 9. A window from the start of current event period up to current time tₚ is considered in this preferred embodiment. Then the set of clusters with a membership higher than the threshold μₘᵢₙ(tₚ) is selected in Eq. 10. Then, final weights are obtained in Eq. 11 computing fuzzy membership of CGM to the selected clusters in the shorter time window [tₚ−t_{w2},tₚ], Finally, the BG prediction Ĝ(t|tₚ) obtained is saved. Optionally, only the first step could be applied, computing the fuzzy membership from a single time window [tₚ−t_{w1},tₚ].

After an event period is finalized and next event period is started, the whole period data is then appended as new data in the cluster, having the highest membership, as well as historical data in the corresponding local model. It is not expected that the profile of the modified CGM data cluster changes too much, since the aspect of the new time series is expected to be similar to the others in the cluster.

It is fundamental to have new series in the cluster since SARIMA models use previous event periods data of the same subset/cluster, for their predictions.

Additionally, it would be interesting to have all this new series stored in the cluster for an eventual online updating of the SARIMA models. The method of the invention could also comprise a step of calculating a “crispness index”, giving information about how much the glucose prediction is produced by a single model, which is provided along with the available glucose predictions and glycemic status estimations at each time instant

Provided that the glucose model is a multi-model, the “crispness index” gives information about how much the model is reliable. Fuzziness is included in the global model to give robustness by the integration of the local models when one of them is not enough to provide the best estimation. Nevertheless, the global model is more reliable if one of the available local models match perfectly the system, i.e. the membership is one for one of the local models and zero for the others.

For quantifying, the normalized distance between the membership values and an equally-distributed-membership vector (1/c, 1 /c, . . . , 1/c) is used. This vector represents the lowest crispness (highest fuzziness), with equal contribution of all local models. Corresponding to a crispness index value of 0. The highest crispness is obtained for a membership of 1 for one of the clusters, and 0 for the others, providing a distance to (1/c, 1 /c, . . . , 1/c) equal to 1/(2(1−1/c)), which should correspond to a crispness index value of 1. The crispness index is thus computed as:

$$CI ( {t}_{p} ) = \frac{1}{2 ( 1 - \frac{1}{c} )} {\sum }_{i = 1}^{c} \vert {γ}_{i} ( {t}_{p} ) - \frac{1}{c} \vert \qquad Eq . 16$$

which corresponds to a normalized Manhattan distance between the membership values and the vector (1/c, 1/c, . . . , 1/c).

The method of the invention could further comprise a step of calculating a “normality index”, giving information about how normal is the actual behavior or if it is an abnormal situation.

The “normality index” is designed to determine whether the measured CGM data in the current event period is similar to historical data represented by the clusters, and represents whether the period trying to estimate is normal or not, in the sense that the behavior trying to forecast, and then could be estimated by a single local model or a combination of them, or is not among said past behaviors, being beyond the past behaviors and then extrapolated by the models. Possibility memberships (γᵢ^{P}(tₚ)) are used in this case, instead of fuzzy memberships. The possibility membership could be computed as described in the equations:

$$\begin{matrix}{u}_{i}^{P} ( {t}_{1} , {t}_{0} ) = \frac{1}{1 + {η ( {d}^{2} ( {[ G ]}_{{t}_{0}}^{{t}_{1}} ( t ) , {[ {v}_{i} ]}_{{t}_{0}}^{{t}_{1}} ( t ) ) )}^{\frac{1}{m - 1}}} & Eq . 17 \\ I = \{ i \vert {u}_{i}^{P} ( {t}_{p} ; {t}_{p} - {t}_{W 1} ) \geq {μ}_{min}^{P} ( {t}_{p} ) \} & Eq . 18 \\ {γ}_{i}^{P} ( {t}_{p} ) = \{ \begin{matrix}{u}_{i}^{P} ( {t}_{p} ; {t}_{p} - {t}_{W 2} ) & for i ∈ I \\ 0 & otherwise\end{matrix} & Eq . 19\end{matrix}$$

In Eq. 17 to 19:
uᵢ^{P}(tᵢ; t₀)∈[0, 1], ∀i
γᵢ^{P}(tₚ)∈[0, 1], ∀i
Σᵢ₌₁^{e}γᵢ^{p}(tₚ)≤1.

Also, d is a distance function (Euclidean distance function between CGM data segment from t₀ to t₁ and the corresponding segment of the cluster prototypes (centers) and m∈[1∞) is the fuzziness parameter.

If the period trying to estimate is abnormal and far away from all the available local models, the distance will be similar to all prototypes then resulting in all the memberships equal when Eq. 12, 14, 15 is applied. The same result is obtained when a period trying to estimate is normal and just in the middle of the local models available. Therefore, Eq. 12 is not useful for detecting abnormal periods.

Using Eq. 17-19 the abnormal period results in very low membership for all the clusters. The sum of all those possibility memberships (Σᵢ₌₁^{e}γᵢ^{p}(tₚ)) computed online provides a measure of the abnormality of the period: the sum of the possibility memberships closer to zero the more abnormal period.

On detecting abnormal behaviors, an extrapolation is performed in predicting BG, and an alert about an abnormal situation is activated, for hardware problems, missed bolus leading to extreme hyperglycemia, unusual exercise leading to hypoglycemia, or any other behavior beyond the past time series available for local models identification. Also, abnormal behavior can provide alerts to the user, highlighting the necessity of new models to be learned due to new user behaviors.

The calculation of the “crispness index” and the “normality index” could be performed by using an online monitoring system.

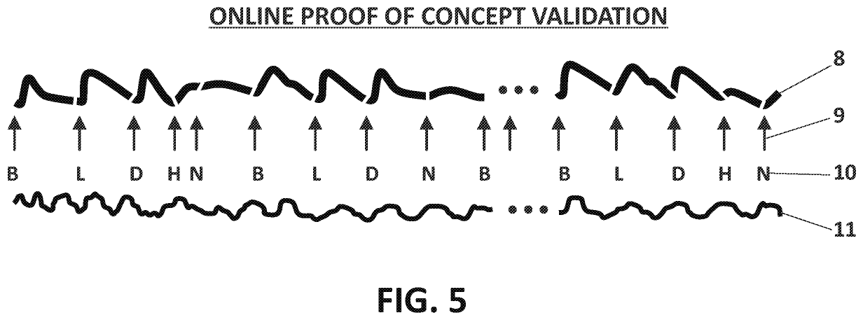

FIG. 5 shows a representation of a validation time series, wherein the CGM data (8) comprises event time stamps (9) and labels (10) (if it is labelled), and also, the exogenous data (11) is added.

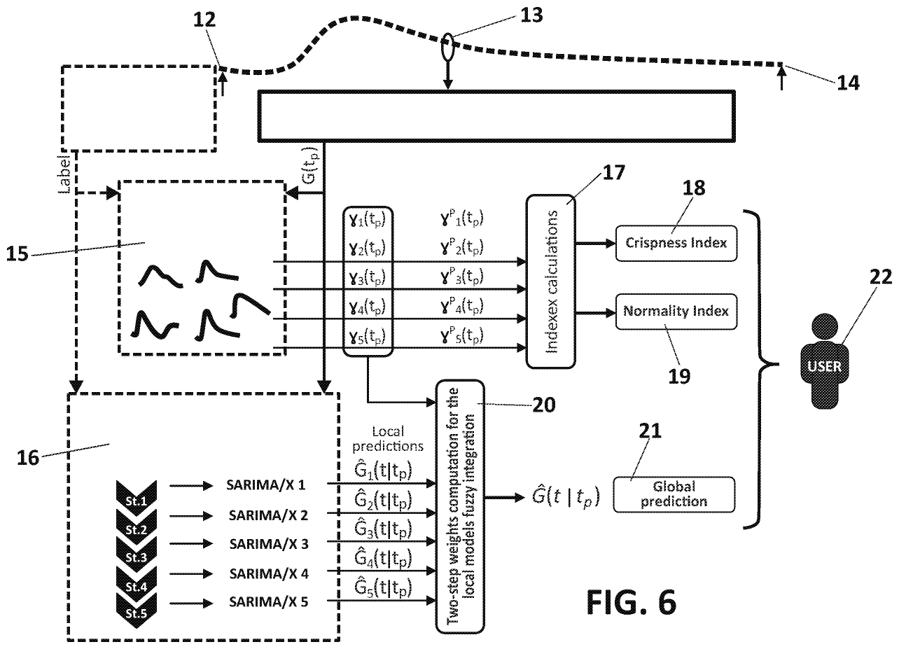

FIG. 6 shows a preferred embodiment of a validation step of the preferred embodiment of the invention. If the data are labelled, then, the labels are used for selecting cluster prototypes (12). Also, at each time tₚ a new glucose measurement G(tₚ) is available (13) and new exogenous data (if provided) from the beggining (12) until the end (14) of the period. From prototypes (15), an indexes calculation (17) is performed, obtaining a crispness index (18) and a normality index (19). The predicting step (16) provides the local models for obtaining local predictions (20) and, then, an integration leads to the global BG prediction Ĝ(t|tₚ) (21).

### EXAMPLE

To analyze the performance of the proposed method, a long-term period of four months data from a virtual patient is used for training purposes (clustering and training of local models). The simulated data were generated using the adult patient number 1 of an extended version of the UVA/Padova simulator with variability sources, and neither exercise not missed boluses were considered. Additionally two 2-month data sets were generated for the same patient for independent validation. In the validation set 1, neither exercise not missed boluses were considered similarly to the training data set. In validation set 2, exercise and missed boluses were added.

In the training data and validation set 1, three daily random meals of 40, 90 and 60 g at 7:00, 14:00 and 21:00 hours were considered, and variability sources include: meal-size variability (coefficient of variance 10%), meal-time variability (standard is deviation 20 min), uncertainty in carbohydrate (CHO) estimation (uniform distribution between −30% and +10%), meal absorption rate (kabs±30%), CHO bioavailability (f±10%), insulin absorption model parameters (kd, ka1, ka2 ±30%), and insulin sensitivity parameters (Vmx, Kp3±30%). In the validation set 2, additionally to all the above, one missed bolus per week was considered, and a weekly pattern of one hour exercise session at 18:00 at 50% intensity on Tuesday, Thursday and Saturday. A variability on the exercise start time (standard deviation 20 min) and duration (coefficient of variance 10%) was considered.

Training data, validation data 1 and validation data 2, included timestamps for breakfast, lunch, dinner and hypo treatments. Labels indicating the event type were also considered.

The method of the invention, in this particular example, starts with the preprocessing of the long-term time series. First, timestamps for night periods are generated from dinner and next-day breakfast reported event, and night period events are labelled accordingly. Then, three subsets of event periods are considered:
Subset 1: Meal periods, including breakfast, lunch and dinner events
Subset 2: Night periods
Subset 3: Hypo treatment periods.

Each subset will be considered independently, applying the method of the invention to each of them, that is, 3 clustering and local model indentification problems are solved. As a result of this pre-processing step, the following event-to-event periods are obtained:
Subset 1: 352 event-to-event periods, with a maximum duration of 106 observations.
Subset 2: 119 event-to-event periods, with a maximum duration of 65 observations.
Subset 3: 18 event-to-event periods, with a maximum duration of 76 observations.

The maximum duration for each subset will determine the seasonality considered for that subset when identifying the local models.

Then, PDSFCM clustering, in combination with the FSI cluster validity index for number of clusters determination, is performed for each subset, after length regularization based on the maximum event-to-event period length for each subset. This resulted in 5 clusters for subset 1, 3 clusters for subset 2 and 2 clusters for subset 3. Once all periods are classified, seasonal time series per cluster per subset are created by concatenating those periods assigned to each cluster.

Additionally, presampling data, i.e. data before the period starts from the CGM time series, is inserted for each concatenated period for an adequate initialization of AR and MA model components. This historical data is needed by the models in a length depending on the models orders. A length of five pre-sampling data is considered enough, in this case.

The four months available training simulated data for the adult patient number 1 of the UVA/Padova simulator with three meals per day and multiple variability sources will be divided into two sets. For data in each cluster, the first 80% of the data is used as a training set for building suitable models, and 20% of the data is used for testing the prediction accuracy for these models.

Model training sets thus consisted of concatenated event periods from the first 80% of elements in each cluster, with enforced seasonalities of 111 for subset 1 (106 samples plus 5 pre-samples), 70 for subset 2 (65+5), and 81 for subset 3 (76+5). The appropriate SARIMA model, including structure and parameters, for each cluster is identified through Box-Jenkins methodology, using the rest 20% of data in each cluster for local model validation.

The metrics used for model forecasting accuracy are root mean squared error (RMSE[mg/dL]) and mean absolute percentage error (MAPE[%]), defined as:

$$CI ( {t}_{p} ) = \frac{1}{2 ( 1 - \frac{1}{c} )} {\sum }_{i = 1}^{c} \vert {γ}_{i} ( {t}_{p} ) - \frac{1}{c} \vert \qquad Eq . 16$$

In Eq. 16 and 17, n is the number of observations, G(i) denote the i^{th} observation, e(i)=G(i)−Ĝ(i) is the forecasting error, and \tilde{G}(i) is a forecast of G(i).

Table I shows the appropriate SARIMA model for each cluster with the average prediction accuracy results for different PH ranging from 15 min to 4 hours, expressed as MAPE (RMSE) computed with Eq. 16-17 as follows: for each event period in the local model validation data (20% of data in a cluster), the average MAPE (RMSE) of the predicted glucose trajectory as time moves along the event period was computed. Then, the average for all event periods in the local model validation data was computed.

TABLE I

| | SARIMA | PH | | | | | |
|---|---|---|---|---|---|---|---|
| Cluster | model | 15 min | 30 min | 60 min | 120 min | 180 min | 240 min |

Subset 1 (meal events)

| | | | | | | | |
|---|---|---|---|---|---|---|---|
| 1 | (3, 0, 1) | 2.35 | 2.83 | 4.70 | 5.77 | 6.62 | 8.39 |
| | (2, 0, 0)₁₁₁ | (2.41) | (3.13) | (6.65) | (7.98) | (8.90) | (10.67) |
| 2 | (3, 0, 1) | 3.35 | 4.33 | 4.95 | 4.19 | 4.34 | 5.09 |
| | (2, 0, 2)₁₁₁ | (4.75) | (6.60) | (8.96) | (8.72) | (9.09) | (10.17) |
| 3 | (2, 0, 3) | 3.22 | 3.79 | 4.81 | 6.00 | 5.69 | 5.86 |
| | (1, 0, 1)₁₁₁ | (3.95) | (5.04) | (8.87) | (13.50) | (13.08) | (13.03) |
| 4 | (2, 0, 3) | 2.48 | 3.93 | 4.94 | 5.26 | 5.32 | 5.92 |
| | (1, 0, 1)₁₁₁ | (3.17) | (5.85) | (9.13) | (12.08) | (12.84) | (14.71) |
| 5 | (2, 0, 1) | 2.49 | 3.24 | 4.51 | 5.63 | 6.07 | 6.16 |
| | (2, 0, 2)₁₁₁ | (3.05) | (4.22) | (7.43) | (10.21) | (10.82) | (10.66) |

Subset 2 (night events)

| | | | | | | | |
|---|---|---|---|---|---|---|---|
| 1 | (2, 0, 1) | 3.12 | 3.68 | 4.56 | 4.42 | 4.88 | 5.18 |
| | (1, 0, 2)₇₀ | (3.32) | (3.92) | (4.76) | (4.72) | (5.16) | (5.89) |
| 2 | (1, 0, 1) | 2.92 | 3.25 | 3.64 | 6.29 | 6.69 | 6.89 |
| | (0, 0, 0)₇₀ | (5.39) | (5.72) | (6.17) | (9.85) | (9.92) | (9.87) |
| 3 | (1, 0, 2) | 2.00 | 3.50 | 3.96 | 4.46 | 4.59 | 4.50 |
| | (2, 0, 1)₇₀ | (2.39) | (4.28) | (4.73) | (5.22) | (5.73) | (5.66) |

Subset 3 (hypo treatment events)

| | | | | | | | |
|---|---|---|---|---|---|---|---|
| 1 | (1, 1, 2) | 0.69 | 1.44 | 5.11 | 5.50 | 7.33 | 8.12 |
| | (1, 1, 0)₈₁ | (0.60) | (1.26) | (7.73) | (8.86) | (12.73) | (14.47) |
| 2 | (4, 0, 0) | 1.78 | 3.68 | 4.37 | 3.08 | —* | —* |
| | (1, 0, 1)₈₁ | (1.53) | (3.85) | (4.73) | (3.85) | (—) | (—) |

*Validation data shorter than PH

In the most challenging scenario of a 4-h PH forecasting period, for meal events (subset 1) a MAPE of 8.39% (RMSE of 10.67 mg/dL) is obtained for cluster 1, 5.09% (10.17 mg/dL) for cluster 2, 5.86% (13.03 mg/dL) for cluster 3,5.92% (14.71 mg/dL) for cluster 4, and 6.16% (10.66 mg/dL) for cluster 5. For night periods (subset 2), 4-h PH forescasting error is 5.18% (5.89 mg/dL) for cluster 1, 6.89% (9.87 mg/dL) for cluster 2, and 4.50% (5.66 mg/dL) for cluster 3. For hypoglycemia treatment periods (subset 3), only cluster 1 data included long enough periods to compute a 4-h PH forecasting, being cluster 2 data shorter than 3 hours. For a 2-h PH, forecasting errors are 5.50% (8.86 mg/dL) for cluster 1 and 3.08% (3.85 mg/dL) for cluster 2. Results for different PHs (15, 30, 60, 120, 180, and 240 minutes) are presented in Table I.

Table II shows the average MAPE (%) and RMSE (mg/dL) values of the global seasonal model (GSM) glucose predictions (after the integration of local models with Eqs. 8 to 11) for the independent 2-month validation data set 1 (same scenario are training data). A value of t_{w1} equal to the time elapsed from the start of the current event was considered. A value of t_{w2} equal to 20 minutes was considered. Finally, the threshold μₘᵢₙ(tₚ) was set to 20% of the maximum membership uᵢ(tₚ;tₚ−t_{w1}) for i=1, . . . , c (with c=5 for subset 1, c=3 for subset 2, and c=2 for subset 3).

TABLE II

| Validation | PH | | | | | |
|---|---|---|---|---|---|---|
| data set 1 | 15 min | 30 min | 60 min | 120 min | 180 min | 240 min |
| Subset 1 | 2.33 | 3.83 | 6.11 | 9.53 | 13.02 | 14.95 |
| (meals) | (3.63) | (6.27) | (10.35) | (15.87) | (21.05) | (24.29) |
| Subset 2 | 2.48 | 3.88 | 5.39 | 6.64 | 7.84 | 8.28 |
| (nights) | (2.73) | (4.41) | (6.31) | (7.97) | (9.49) | (10.25) |
| Subset 1 | 2.44 | 3.79 | 5.12 | 7.21 | 9.75 | 13.99 |
| (hypotreat.) | (3.15) | (5.32) | (8.18) | (12.21) | (16.19) | (23.98) |
| Global | 2.36 | 3.84 | 6.00 | 9.18 | 12.62 | 14.84 |
| | (3.49) | (5.99) | (9.78) | (14.96) | (20.18) | (24.06) |

The results reported in Table II allow us to conclude that the GSM, when challenged with data similar to the one represented by the local models, exhibits high prediction accuracy for larger PHs (up to 240-min-ahead prediction), therefore, GSM could be helpful to allow the diabetic patients and glucose management systems anticipate therapeutic decisions.

TABLE III

| Validation | PH | | | | | |
|---|---|---|---|---|---|---|
| data set 2 | 15 min | 30 min | 60 min | 120 min | 180 min | 240 min |
| Subset 1 | 3.08 | 5.12 | 8.40 | 13.46 | 17.45 | 18.55 |
| (meals) | (4.89) | (8.53) | (14.32) | (22.50) | (27.92) | (29.48) |
| Subset 2 | 2.72 | 4.47 | 6.71 | 9.60 | 12.48 | 12.76 |
| (nights) | (3.75) | (6.37) | (9.70) | (13.75) | (17.63) | (17.51) |
| Subset 3 | 4.94 | 8.86 | 13.95 | 17.29 | 26.74 | — |
| (hypotreat.) | (4.25) | (7.80) | (12.69) | (17.24) | (25.31) | (—) |
| Global | 3.14 | 5.24 | 8.39 | 12.99 | 16.95 | 18.39 |
| | (4.66) | (8.13) | (13.52) | (21.19) | (26.89) | (29.16) |

Table III shows the average MAPE (%) and RMSE (mg/dL) values of global glucose predictions for the independent 2-month validation set 2, where missed insulin boluses at mealtime happened once per week and exercise 3 times per week, which are events not present in the training data. It is thus expected a higher error, as observed in Table III, which amounts to about 4% increase for long-term PH. However, forecasting errors are still considered globally small, with 12.99% MAPE for a 120-min-ahead prediction, 16.95% for 180-min PH, and 18.39% for 240-min PH.

The integration process is updated online for each validation event period in order to get the online BG prediction by following the steps of:
a) Selecting the subset of clusters and local models corresponding to the received event label (subset 1 for a meal event, subset 2 for night event, subset 3 for a hypoglycemia treatment event)
b) calculating the membership to the selected set of c clusters of the CGM measurements up to current time for the current event period;
c) combining the predictions of the c local models using the memberships calculated in step b);
d) repeating steps b) and c) every new CGM measurement until the period is finished (the next event timestamp is received);
i) adding the finished event period as a new element of the cluster having the highest membership, and the historical data of the corresponding local model; and
e) repeating the previous steps with the new event period and calculate the mean prediction error for the whole validation data set (i.e. for 180 meal periods, 59 night periods and 15 hypoglycemia treatment periods in validation set 1; and for 180 meal periods, 59 night periods and 53 hypoglycemia treatment periods in validation set 2).

In addition to the glucose predictions value, a crispness index and a normality index were calculated at each time instant. The crispness index, using for its calculation the changing with time fuzzy memberships, is high (close to one) when a single model represents the behavior for some time, and very low (close to zero) when interpolation among all clusters is needed with equal weight. In a normal period, a high crispness index will be an indication of trust in the prediction. The normality index, using for its calculation the changing with time possibilistic memberships, is high (close to one) when a lot of the available models can represent the behavior and very low (close to zero) when none of them represent the behavior and, therefore, an abnormal situation is presented. A threshold can be set on the normality index to trigger a warning of abnormal response observed (0.2 or lower in this example).

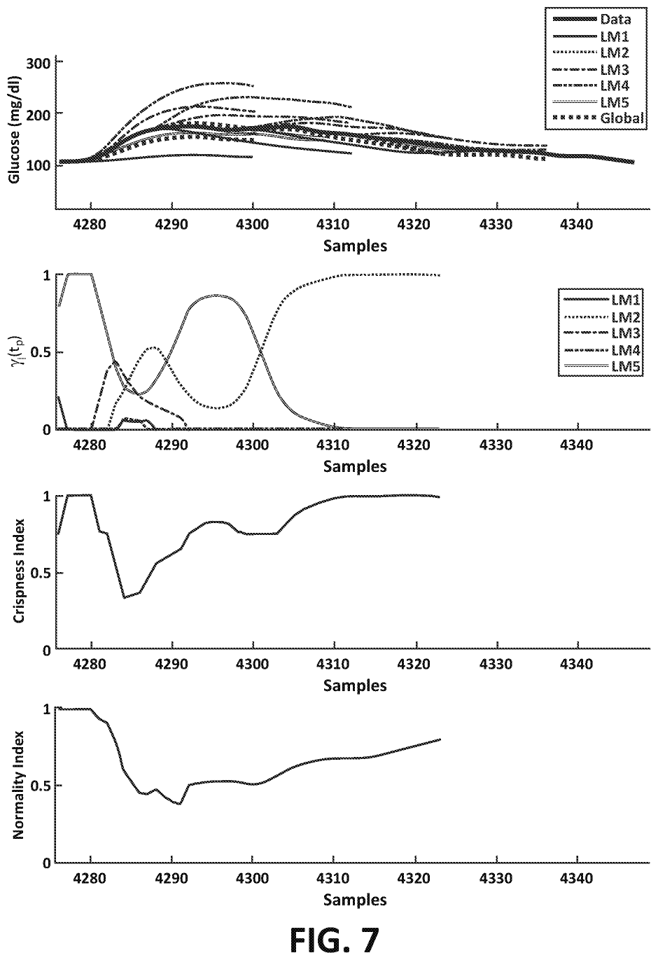

FIG. 7 shows the whole information available at each instant for a meal event, including five (Ĝᵢ(t|tₚ)) local estimations, a global estimation through the integration process, five fuzzy memberships (γᵢ(tₚ)) determining the weighting of the local estimations to get the global estimation, a crispness index and a normality index.

Two cases can be devised in the plots with respect to crispness index:
i. the more local models are used for global prediction the lower the crispness index is, as shown between samples 4280 and 4290;
ii. when the same local model is the mainly used for the global prediction, then the crisp index increases, as shown up to sample 4280, where the model 5 represents well the behavior, and from sample 4310, where model 2 represents well the behavior;

Regarding normality index, as shown in the FIG. 7, a value of one is obtained up to sample 4280, indicating the recent CGM data (20 min window) follows a profile commonly seen in historical data, in this case the prototype of cluster 5. The same happens from sample 4310, with respect to cluster 2. In between, normality index decreases, explanation of recent CGM data requires the combination of prototypes from several clusters. However, normality values are above the set threshold of 0.2. Therefore, no abnormal behavior (far enough from the cluster prototypes) is devised.

Abnormal states could be detected through the normality index. In the case of abnormal behavior time series, the MAPE and RMSE will be high, since this kind of abnormal behavior was not included for model identification (in the training data). This was illustrated in Table III, with validation dataset 2 including missed boluses and exercise, not present in data used for models training.

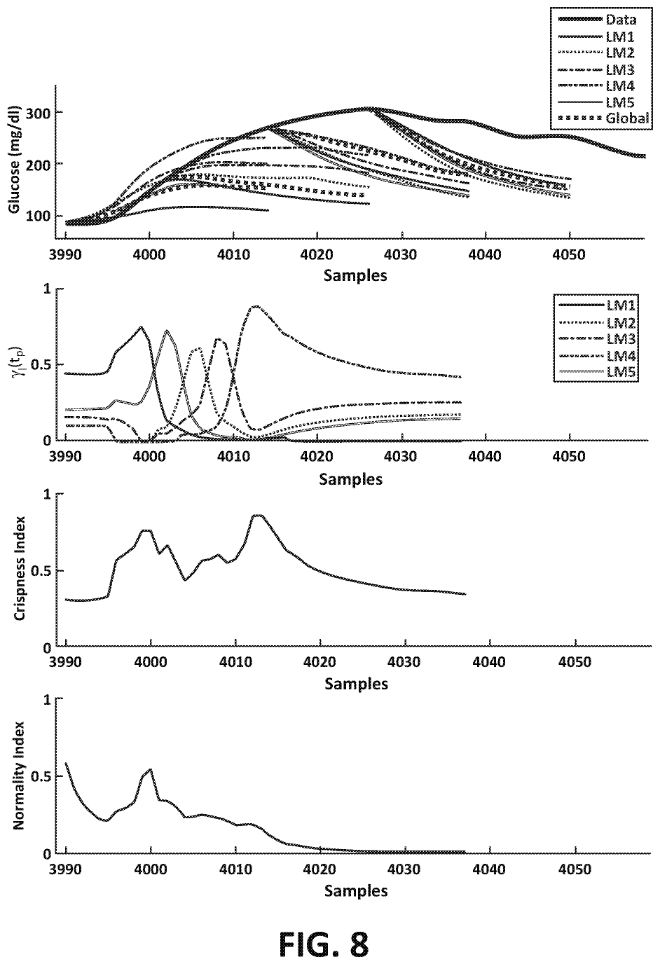

FIG. 8 is included to illustrate how the system behaves in the case of abnormal behaviors. As shown, predictions are very poor and the trust index is low compared to FIG. 7 in different moments with contributions from many models to the global glucose prediction. This is combined with a very low normality index, indicating that predictions are a result of extrapolation, since recent CGM data cannot be explained by any of the cluster prototypes.

Thus, the abnormal state (response to an unexpected missed bolus) can be detected through the normality index, where the lower values below 0.2 indicate an abnormal state. This means that no local model can represent the behavior and therefore, an alarm for the user can be launched to take corrective actions before upcoming extreme hyperglycemia is reached. An artificial pancreas and decision support system can also take automatic actions when this alarm is received.

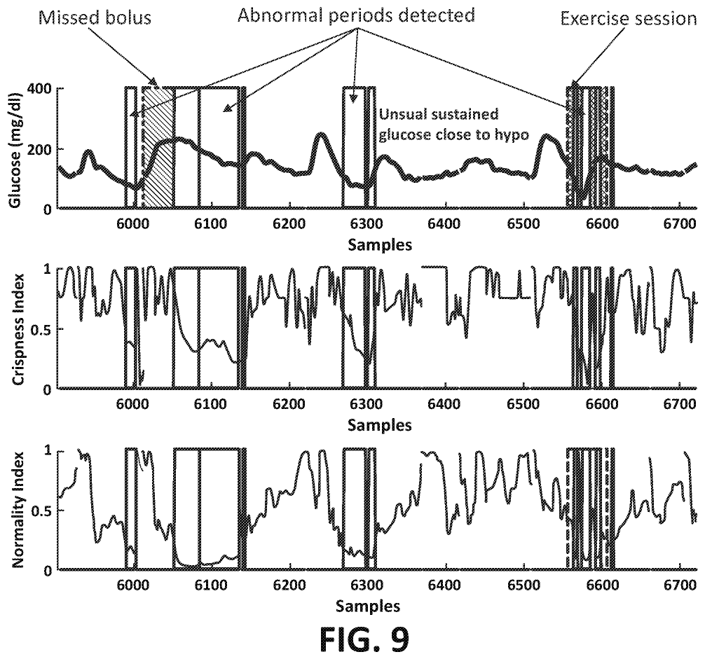

To illustrate the detection of abnormal states, FIG. 9 is provided. Marked boxes indicate the periods detected as abnormal, corresponding to a normality index below or equal to 0.2. Shaded box indicates a postprandial period with a missed bolus. Meshed box indicate an exercise session including the 3-hour period post-exercise with altered insulin sensitivity. Sustained hyperglycemia due to the missed bolus, as compared to a regular meal, is detected as an abnormal period. Rapid decline of glucose due to the exercise and posterior dinner response (with altered insulin sensitivity and mismatch insulin bolus) is also detected as abnormal state.

Additionally a period around sample 6300 is also detected as abnormal, corresponding to an unusual sustained glucose close to hypoglycemia in a late postprandial response.

As demonstrated, the crispness index is useful for measuring goodness of a single cluster to represent the recent CGM data giving then confidence in the estimation, especially when analyzed in combination with the normality index. The normality index is useful for detecting the abnormal behavior/states allowing for the triggering of corrective actions if needed to mitigate risks for the patient.

1. Method for predicting blood glucose based on seasonal local models, comprising the steps of:
providing raw data time series, comprising a record of measured blood glucose (BG) data;
preprocessing the raw data time series for obtaining event periods by:
dividing the raw data time series into event periods, by setting timestamps of main meal events, so that an event period starts at a timestamp of said event and ends on the timestamp of the next event and wherein the event period after last meal in day is split in two period events;
enforcing seasonality of event periods by expanding fictitiously, adding not a number values after the last measured BG value, the duration of the event periods, until the duration of each event period is equal to the duration of the period event having the maximum duration (s);
clustering event periods by using techniques for clustering incomplete data, which partitions data into c cluster prototypes, being the cluster defined by a centroid and distance measures;
identifying a set of c seasonal local models, each one representing glucose time series in a cluster at each time step (tp),
predicting the blood glucose for a desired prediction horizon PH by using the set of seasonal local models, each one representing a cluster (local glucose predictions),
integrating the local glucose predictions for obtaining a real-time BG prediction for a desired prediction horizon, through real-time membership-to-cluster estimation; and
saving a BG prediction Ĝ(t|tₚ), t in [tₚ,tₚ+PH].

2. Method according to claim 1, further comprising a step of dividing the subset of event periods into different subset groups of data using data labels, wherein the steps of clustering and predicting blood glucose is applied to each labeled subset group.

3. Method according to claim 1, wherein in the step of dividing the raw data time series into event periods, additional events are included including at least one of hypoglycemia treatment and exercise session.

4. Method according to claim 1, wherein the step of clustering event periods is performed by using a partial distance strategy fuzzy C-Means clustering (PDSFCM) algorithm, which partitions data into c cluster prototypes by minimizing the equation:
$${I}_{m} ( U , V ; X ) = {\sum }_{i = 1}^{c} {\sum }_{j = 1}^{n} {u}_{ij}^{m} {d}_{ij}^{2} ( {x}_{j} , {v}_{i} )$$
wherein, X={x1, x2, . . . , xn} denotes regularized-length event periods extracted from raw data time series, V=(v1, v2, . . . , vc) is a vector of unknown cluster prototypes vᵢ∈ [symbol] ^{p}, d is a distance function representing the partial distance between the raw data time series and cluster prototypes and m∈[1, ∞) is a fuzziness parameter, U=[uᵢⱼ] with i=1, 2, . . . , c, and, j=1, 2, . . . , n is a matrix of memberships wherein:
uᵢⱼ∈[0, 1];
Σᵢ₌₁^{e}uᵢⱼ=1∀j; and
0<Σⱼ₌₁ⁿuᵢⱼ<n, ∀i.

5. Method according to claim 1, wherein the step of predicting the blood glucose of each time series is performed by using a SARIMA model, defined by the equation:
G(t)=α+w(t)
ϕₚ(z⁻¹) [symbol] _{P}(z^{-s})∇ₛ^{D}∇^{d}w(t)=θ_{q}(z⁻¹)θ_{Q}(z^{-s})ε(t)
wherein G(t) is the glucose concentration at time t, α is a constant term, called intercept, ∇ is the backward difference operator, defined as:
∇w(t)=w(t)−w(t−1)
d is the non-seasonal integration order of the time series, D is the seasonal integration order of the time series, ∇s is the seasonal backward difference operator, defined by:
∇ₛw(t)=w(t)−w(t−s)
ε(t) is an stochastic error following a white noise process defined by:
ε(t)˜WN(0, σ²)
and ϕₚ(z⁻¹), [symbol] _{P}(z^{-s}), θ_{q}(z⁻¹) and θ_{Q}(z^{-s}) are polynomials in the lag (back-shift) operator z-1 of degree p, q, P, and Q, respectively, defined as:
ϕₚ(z⁻¹)=1−ϕ₁z⁻¹−ϕ₂z⁻²− . . . −ϕₚz^{-p}
Φ_{P}(z^{-s})=1−Φₛz^{-s}−Φ₂ₛz^{-2s}− . . . −Φ_{Ps}z^{-Ps}
θₚ(z⁻¹)=1+θ₁z⁻¹+θ₂z⁻²+ . . . +θ_{q}z^{-q}
θ_{Q}(z^{-s})=1+θₛz^{-s}+θ₂ₛz^{-2s}+ . . . +θ_{Qs}z^{-Qs}

6. Method according to claim 1, wherein the step of integrating the SARIMA glucose models is performed by a time-varying weighting of the set of glucose predictions given by the seasonal local models, defined by the equations:
$$\hat{G} ( t \vert {t}_{p} ) = {\sum }_{i = 1}^{c} {γ}_{i} ( {t}_{p} ) {\hat{G}}_{i} ( t \vert {t}_{p} )$$
where tₚ is the time at which the prediction is computed, t is the time of the predicted value, in the time interval [tₚ,tₚ+PH], Ĝ(t|tₚ) is the predicted glucose by local model i, for i={1, 2, . . . , c},) is a fuzzy membership corresponding to the weight of model i at time tp, and Ĝ(t|tₚ) is the final predicted glucose value for future time t, computed at current time tₚ, and wherein the time-varying weights γᵢ(tₚ), i=1, . . . , c, are computed from the membership value of CGM measurements to each cluster i, with respect to a time window [t₀,t₁], denoted as uᵢ(t₁; t₀), and defined by:
$${u}_{i} ( {t}_{1} ; {t}_{0} ) = \frac{1}{{\sum }_{i = 1}^{c} {( \frac{{d}^{2} ( {[ G ]}_{{t}_{0}}^{{t}_{1}} ( t ) , {[ {v}_{i} ]}_{{t}_{0}}^{{t}_{1}} ( t ) )}{{d}^{2} ( {[ G ]}_{{t}_{0}}^{{t}_{1}} ( t ) , {[ {v}_{i} ]}_{{t}_{0}}^{{t}_{1}} ( t ) )} )}^{\frac{1}{m - 1}}}$$
where [G]ₜ_{₀}^{t}^{₁}(t) is the segment of CGM data from t₀ to t₁, [vᵢ]ₜ_{₀}^{t}^{₁}(t) is the segment of the prototype for cluster i from t₀ to t₁, and d is the distance measured.

7. Method according to claim 6, wherein the time window [t₀,t₁] is defined as the time interval from the timestamp of the last event (tₑₖ) up to current time (tₚ), so that:

8. Method according to claim 6, wherein the time-varying weights γᵢ(tₚ), i=1, . . . , c, are computed in a two-step procedure, wherein in a first step, clusters with a high enough contribution in a first time window [tₚ−t_{w1},tₚ] are selected, as defined by the index set:
I={i|uᵢ(tₚ;tₚ−t_{w1})≥μₘᵢₙ(tₚ)}
and wherein t_{w1}=tₑₖ wherein is chosen, and in a second step, selected clusters contributions are computed, with respect a second shorter time window [tₚ−t_{w2},tₚ], so that t_{w2}t_{w1}, as defined by:
$${γ}_{i} ( {t}_{p} ) = \{ \begin{matrix}{u}_{i} ( {t}_{p} ; {t}_{p} - {t}_{W 2} ) & for i ∈ I \\ 0 & otherwise\end{matrix}$$

9. Method according to claim 1, wherein exogenous inputs are provided and wherein a SARIMAX model is used for predicting the blood glucose of each time series, defined by:
$$G ( t ) = α + {\sum }_{i = 1}^{{n}_{x}} {η}_{i , {r}_{i}} ( {z}^{- 1} ) {X}_{i} ( t ) + w ( t )$$
$${ϕ}_{p} ( {z}^{- 1} ) {ϕ}_{p} ( {z}^{- s} ) {∇}_{s}^{D} {∇}_{ }^{d} w ( t ) = {θ}_{q} ( {z}^{- 1} ) {θ}_{Q} ( {z}^{- 0} ) ɛ ( t )$$
where nₓ is the number of exogenous inputs, Xᵢ is the exogenous input i at time t and η_{i,r}(z⁻¹), i=1, . . . , nₓ are polynomials in the lag operator z⁻¹ of degree rᵢ, defined as:
η_{i,r}_{ᵢ}(z⁻¹)=η_{i,0}+η_{i,1}z⁻¹+η_{i,2}z⁻²+ . . . +η_{i,r}_{ᵢ}z^{-r}^{ᵢ}

10. Method according to claim 1, wherein Xie-Beni index (XBI) or Fukuyama-Sugeno index (FSI) are used for determining the optimal number of clusters.

11. Method according to claim 1, further comprising a phase of identifying an optimal seasonal model previous to the step of predicting the blood glucose of each time series by means of the Box-Jenkins methodology, including steps of checking for stationarity or non-stationarity seasonal models and differencing the data, if necessary; identifying and selecting an appropriate model structure; estimating parameters of the chosen model; diagnostic checking of the chosen model; and forecasting, and re-identifying a new model, if necessary.

12. Method according to claim 1, wherein the step of integrating SARIMA glucose models for obtaining a real-time BG prediction further comprises:
a) receiving a new event time stamp, and if the event is labelled, an event type label;
b) receiving a CGM measurement;
c) if the event is labelled, selecting a set of c clusters and corresponding local models for that event type, obtained in the clustering and predicting steps;
d) calculating the membership to the selected set of c clusters of the CGM measurements up to current time for the current event period;
e) combining the predictions of the c local models using the memberships calculated in step d);
f) repeating steps d) and e) every new CGM measurement until the period is finished;
g) adding the finished period as a new element of the cluster, having the highest membership, and the historical data of the corresponding local model; and
h) repeating the previous steps with the new event period.

13. Method according to claim 1, further comprising an step of calculating a crispness index, which gives a measure of how much the glucose prediction is produced by a single model, by applying a Manhattan distance, normalized to the interval [0,1], between a vector composed by the membership values to the c clusters and an equally-distributed-membership vector (1/c, 1 /c, . . . , 1/c) representing the lowest crispness, defined as
$$CI ( {t}_{p} ) = \frac{1}{2 ( 1 - \frac{1}{c} )} {\sum }_{i = 1}^{c} \vert {γ}_{i} ( {t}_{p} ) - \frac{1}{c} \vert \qquad$$

14. Method according to claim 1, further comprising the step of calculating a normality index, which gives a measure of how much the measured CGM data in the current event period is similar to historical data represented by the clusters, by:
calculating possibility memberships
summing up the possibility memberships and normalizing to the interval [0,1],
determining the normality degree of the period:
if the sum of possibility memberships is greater than a defined threshold (thr_{Nl}), then it is normal,
if the sum of possibility memberships is lower than thr_{Nl}, then it is abnormal.

15. System for controlling blood glucose automatically based on seasonal local models, comprising one or more CGM sensors; and a control unit, connected to the CGM sensors and adapted to perform the following steps:
providing raw data time series, comprising a record of measured blood glucose (BG) data;
preprocessing the raw data time series for obtaining event periods by:
dividing the raw data time series into event periods, by setting timestamps of main meal events, so that an event period starts at a timestamp of said event and ends on the timestamp of the next event and wherein the event period after last meal in day is split in two period events;
enforcing seasonality of event periods by expanding fictitiously, adding not a number values after the last measured BG value, the duration of the event periods, until the duration of each event period is equal to the duration of the period event having the maximum duration (s);
clustering event periods by using techniques for clustering incomplete data, which partitions data into c cluster prototypes, being the cluster defined by a centroid and distance measures;
identifying a set of c seasonal local models, each one representing glucose time series in a cluster at each time step (tp),
predicting the blood glucose for a desired prediction horizon PH by using the set of seasonal local models, each one representing a cluster (local glucose predictions),
integrating the local glucose predictions for obtaining a real-time BG prediction for a desired prediction horizon, through real-time membership-to-cluster estimation; and
saving a BG prediction \tilde{G}(t|tₚ), t in [tₚ,tₚ+PH].

16. System according to claim 15, further comprising a pump for delivering insulin connected to the control unit, and wherein said control unit is further configured to calculate the amount of insulin to provide according to the calculated BG prediction.

17. System according to claim 15, further comprising a communication module connected to the control unit, to connect to an external device, wherein the control unit is further configured to calculate a crispness index by applying a Manhattan distance, normalized to the interval [0,1], between a vector composed by the membership values to the c clusters and an equally-distributed-membership vector (1/c, 1/c, . . . , 1/c) representing the lowest crispness, defined as:
$$CI ( {t}_{p} ) = \frac{1}{2 ( 1 - \frac{1}{c} )} {\sum }_{i = 1}^{c} \vert {γ}_{i} ( {t}_{p} ) - \frac{1}{c} \vert \qquad$$

18. System according to claims 15, further comprising an alert module connected to the control unit, to activate an alarm when a hypoglucemia or hyperglucemia are expected, wherein the control unit is further configured to calculate a normality index following the steps of:
calculating possibility memberships
summing up the possibility memberships and normalizing to the interval [0,1],
determining the normality degree of the period:
if the sum of possibility memberships is greater than a defined threshold (thr_{Nl}), then it is normal,
if the sum of possibility memberships is lower than thr_{Nl}, then it is abnormal.
